(19) United States
(12) Patent Application Publication
(10) Pub. No.: US 2023/0364075 A1
(43) Pub. Date: Nov. 16, 2023

(54) MULTIPARTICULATE DOSAGE FORMS COMPRISING DEUTETRABENAZINE

(71) Applicant: AUSPEX PHARMACEUTICALS, INC., Parsippany, NJ (US)

(72) Inventors: Mayank JOSHI, Parsippany, NJ (US); Parag SHAH, Parsippany, NJ (US); Soumen PATTANAYEK, Parsippany, NJ (US); Divyang PATEL, Parsippany, NJ (US); Sandeep PANDITA, Parsippany, NJ (US)

(21) Appl. No.: 18/245,565

(22) PCT Filed: Sep. 17, 2021
(86) PCT No.: PCT/US2021/050785

§ 371 Date: Mar. 16, 2023

Related U.S. Application Data
(60) Provisional application No. 63/079,786, filed on Sep. 17, 2020.
(60) Provisional application No. 63/091,064, filed on Oct. 13, 2020.

(51) Int. Cl. A61K 31/4745; A61K 9/14; A61K 9/16; A61K 9/50

(52) U.S. Cl. CPC A61K 31/4745; A61K 9/14; A61K 9/1676; A61K 9/1623; A61K 9/1617; A61K 9/1652; A61K 9/1641; A61K 9/5026; A61K 9/501; A61K 9/5042; A61K 9/5031; A61K 9/5015; A61K 9/1694

(57) ABSTRACT

Provided herein are controlled release multiparticulate dosage forms containing deutetrabenazine for use in the treatment of, e.g., hyperkinetic movement disorders. When orally administered to a subject on a once-daily basis, the dosage forms provide a favorable pharmacokinetic profile.

### CROSS REFERENCE TO RELATED APPLICATIONS

This application claims the benefit of U.S. Provisional Application No. 63/079,786, filed Sep. 17, 2020, and U.S. Provisional Application No. 63/091,064, filed Oct. 13, 2020, the entireties of which are incorporated herein by reference.

### TECHNICAL FIELD

The present disclosure pertains to multiparticulate dosage forms, manufacturing processes and methods of use of the multiparticulate dosage forms for treating hyperkinetic movement disorders deriving from conditions including Huntington's disease, tardive dyskinesia, levodopa-induced dyskinesia and dyskinesia in cerebral palsy.

### BACKGROUND

Deutetrabenazine ((RR,SS)-1,3,4,6,7,11b-hexahydro-9,10-di(methoxy-D3)-3-(2-methylpropyl)-2H-benzo[a]quinolizin-2-one) is a vesicular monoamine transporter type 2 (VMAT2). The biologically active metabolites formed from deutetrabenazine (alpha-dihydrodeutetrabenazine [α-deuHTBZ] and beta-dihydrodeutetrabenazine [β-deuHTBZ]), together identified as “deuHTBZ”, are potent inhibitors of VMAT2 binding. Deutetrabenazine exhibits an increased half-life of its active metabolites, relative to tetrabenazine (e.g., U.S. Pat. No. 8,524,733).

Deutetrabenazine (deu-TBZ) is approved by the U.S. Food and Drug Administration under the tradename AUSTEDO® for the treatment of chorea (involuntary muscle movements) associated with Huntington's disease (HD) and for the treatment of tardive dyskinesia (TD) in adults. AUSTEDO® dosage forms are orally administered twice-daily (bid), for total daily dosages of 12 mg or above of deutetrabenazine.

Several factors affect gastrointestinal absorption of orally administered drugs including solubility of the drug at various pH and the rate at which drug is released from the dosage form. Drug release rates for oral dosage forms are typically measured as rate of dissolution in vitro, i.e., a quantity of drug released from the dosage form per unit time in, for example, an FDA approved system. Such systems include, for example, United States Pharmacopeia (USP) dissolution apparati I, II and III.

The therapeutic window of a drug is the period when the plasma drug concentration is within the therapeutically effective plasma drug concentration range. Because the plasma drug concentration declines over time, however, multiple doses of drug dosage form must be administered at appropriate intervals to ensure that the plasma drug concentration remains within or, again rises to, the therapeutic window. At the same time, however, there is a need to avoid or minimize plasma drug concentrations that result in undesirable side effects.

Several dosage forms comprising deutetrabenazine are disclosed in U.S. Pat. No. 9,296,739. A dosage form that can deliver deutetrabenazine in a controlled manner over an extended period would enable a more advantageous dosing regimen, e.g., one that would permit once-daily (“qd”) administration while maintaining the treatment effects currently realized by AUSTEDO®. A need exists for such alternative dosage forms.

### SUMMARY

Disclosed herein are controlled release multiparticulate dosage forms for once daily oral administration of deutetrabenazine to a subject in need thereof. The dosage forms, which may be packaged for example, in a capsule or pharmaceutical sachet package, are suitable for the target population.

Provided herein are controlled release oral dosage forms for once daily administration of deutetrabenazine comprise a population of sustained release beads; wherein the sustained release beads comprise a core comprising an amount of deutetrabenazine and a pharmaceutically acceptable excipient, and further comprising a pH-independent polymer coat, a pH-dependent polymer coat or a pH-independent polymer coat further coated with a pH-dependent polymer coat. The core may be one of several forms, for example a) immediate release granules, immediate release pellet or immediate release tablet comprising the deutetrabenazine and the pharmaceutically acceptable excipient or b) an inert particle coated with a dispersion of the deutetrabenazine and the pharmaceutically acceptable excipient. In some embodiments, the dosage form includes a population of the sustained release beads.

In other embodiments, the dosage form includes the population of the sustained release beads and a population of immediate release beads; wherein the population of immediate release beads comprises a) immediate release granules, immediate release pellet or immediate release tablet comprising an amount of deutetrabenazine and a pharmaceutically acceptable excipient or b) an inert particle coated with an amount of deutetrabenazine and a pharmaceutically acceptable excipient. In some embodiments of the dosage form, the core per se of the sustained release particles serves as the population of immediate release beads. Therefore, in some embodiments, the amount of deutetrabenazine and/or the pharmaceutically acceptable excipient are the same in the core of the sustained release beads and in the immediate release beads. However, the amount of deutetrabenazine and/or the pharmaceutically acceptable excipient may be different in the core of the sustained release beads and in the immediate release beads. In some embodiments, the composition of the pharmaceutically acceptable excipient may be same or different in the core of the sustained release beads and in the immediate release bead.

In some embodiments, the deutetrabenazine of the core of the sustained release beads, or present in the immediate release beads is nanonized deutetrabenazine and has a median particle size of 0.02 to 2.0 micron, or 0.02 to 0.9 micron, or 0.05 to 0.5 micron, or 0.1 to 2.0 micron, or 0.1 to 1.6 micron, or 0.2 to 1.6 micron, or 0.15 to 1.2 micron, or 0.15 to 1.0 micron. In some embodiments, the deutetrabenazine has a particle size distribution characterized by a D90 of about 0.8 to about 1.6 micron; a particle size distribution characterized by a D50 of about 0.1 to about 0.6 micron, or about 0.2 to about 0.6 micron; a particle size distribution characterized by a D10 of about 0.1 to about 0.2 micron. In some embodiments, the deutetrabenazine has a particle size distribution characterized by a D90 of about 0.8 to 1.6 micron, a D50 of about 0.2 to about 0.6 micron and a D10 of about 0.1 to about 0.2 micron. In various embodiments, the deutetrabenazine in the core of the sustained release beads or in the immediate release beads, is present in at a concentration of 5 wt %-80 wt %, or 10 wt %-80 wt %, or 10 wt %-70 wt %, 20 wt %-60 wt %, 5 wt % 30 wt %, or 50 wt %-80 wt % of the weight of the core or of the immediate release bead, respectively.

The deutetrabenazine is present in the core or in the immediate release beads together with a pharmaceutically acceptable excipient independently comprising any one of an antioxidant, a binder, a filler, a surfactant, an anti-foaming agent or combinations thereof. In some embodiments, the pharmaceutically acceptable excipient independently comprises an antioxidant, a binder, a filler, a surfactant, and an anti-foaming agent.

In some embodiments, the pharmaceutically acceptable excipient comprises an antioxidant, which may be a water-insoluble antioxidant. The water-insoluble antioxidant comprises butylated hydroxytoluene (BHT), butylated hydroxyanisole (BHA), propyl gallate, 6-ethoxy-1,2-digydro-2,2,4-trimethylquinoline (ethoxyquin), nordihydroguaiaretic acid (NDGA), sodium metabisulfite (SMB), a tocopherol or combinations thereof. In some embodiments, the water-insoluble antioxidant comprises butylated hydroxytoluene (BHT), butylated hydroxyanisole (BHA) or a combination thereof. The water-insoluble antioxidant may be present in the core or in the immediate release bead at a concentration of 0.1 wt %-1.0 wt % of the weight of the core or the immediate release bead, respectively.

In some embodiments, the pharmaceutically acceptable excipient comprises a binder. The binder may be selected from the group consisting of a water-soluble binder, a water-insoluble binder and combinations thereof. In some embodiments, the binder comprises a water-soluble binder, which includes hydroxypropyl cellulose, hydroxypropyl methylcellulose, polyvinyl pyrrolidone, polyvinyl alcohol, a polyacrylic acid polymer, polyether, carbohydrate polymer (natural or synthetic) or combinations thereof. In some embodiments, the binder comprises a water-insoluble polymer, which includes crospovidone, copovidone, microcrystalline cellulose, croscarmellose sodium, starch, sodium starch glycolate, colloidal silica, silica, ethyl cellulose, lactic acid polymer, a lactic acid and glutamic acid copolymer, polyvinyl acetate or combinations thereof. In some embodiments, the binder comprises a polyether, including polyethylene glycol (PEG). The binder may be present in the core or in the immediate release bead at a concentration of 0.5 wt %-10.0 wt % of the weight of the core or the immediate release bead, respectively.

In some embodiments, the pharmaceutically acceptable excipient comprises a filler. The filler may be a saccharide, a disaccharide, a polysaccharide, a polyalcohol, microcrystalline cellulose, natural and synthetic gums, pregelatinized starch, polyvinylpyrrolidone, cellulose derivatives, dibasic calcium phosphate, kaolin, inorganic salts, calcium carbonate, sodium bicarbonate, sodium carbonate, and combinations thereof. In some embodiments, the filler comprises microcrystalline cellulose, a saccharide or a combination thereof. In some embodiments, the saccharide is lactose. The filler may be present in the core or in the immediate release bead at a concentration of 5.0 wt %-50.0 wt % of the weight of the core or the immediate release bead, respectively.

In some embodiments, the pharmaceutically acceptable excipient comprises a surfactant. The surfactant may include sodium lauryl sulfate, sodium dodecyl sulfate, sodium laureth sulfate, docusate sodium, polysorbate, tween, polyoxyethylene 15 hydroxy stearate, polyoxyethylene castor oil derivatives, polyoxyethylene stearates, sorbitan fatty acid esters, polyoxyethylene alkyl ethers, polyoxyethylene nonylphenol ether or combinations thereof. In some embodiments, the surfactant includes sodium lauryl sulfate. The surfactant may be present in the core or in the immediate release bead at a concentration of 2.0 wt %-12.0 wt % of the weight of the core or the immediate release bead, respectively.

In some embodiments, the pharmaceutically acceptable excipient comprises an anti-foaming agent. The anti-foaming agent may include insoluble oils, polydimethylsiloxanes and other silicones, certain alcohols, stearates, glycols and combinations thereof, preferably simethicone, dimethicone, tilactase or peppermint oil. The anti-foaming agent may be present in the core or in the immediate release bead at a concentration of 0.3 wt %-3.0 wt % of the weight of the core or the immediate release bead, respectively.

The core of the sustained release beads may be coated with a coating which may be a pH-independent polymer coat and/or a pH-independent polymer coat. In some embodiments, the sustained release beads include a pH-independent polymer coat. The pH-independent polymer coat may be a cellulose acetate, a mixture of cellulose acetates, ethylcellulose or a mixture of ethylcellulose and polyethylene glycol. In some embodiments, the pH-independent polymer coat comprises ethylcellulose. In some embodiments, the pH-independent polymer coat comprises cellulose acetate. In some embodiments, the pH-independent polymer coat comprises a mixture of cellulose acetate NF 398-10 and cellulose acetate 320S. In some embodiments, the pH-independent polymer coat comprises a mixture of cellulose acetate and polyethylene glycol. The sustained release beads may further include a pH-dependent polymer coat coating the pH-independent polymer coat.

In some embodiments, the sustained release beads include a pH-dependent polymer coat coating the core. In some embodiments, the pH-dependent polymer coat is formulated to dissolve at a pH of about 5.0-7.0, for example in the upper small intestine of a human subject. The pH-dependent polymer coat may be methacrylic acid-ethyl acrylate copolymer, hydroxypropylmethyl cellulose phthalate (HPMCP), alginates, carboxymethylcellulose, or a combination thereof. In some embodiments, the pH-dependent polymer coat comprises methacrylic acid-ethyl acrylate copolymer.

In some embodiments, the pH-dependent polymer coat is formulated to dissolve at a pH above 7.0, for example in the large intestine or colon of a human subject. In that case, the pH-dependent polymer coat may include cellulose acetate phthalate, hydroxypropyl methylcellulose phthalate, hydroxypropyl methylcellulose succinate, polyvinyl acetate phthalate, pH-sensitive methacrylic acid-methyl methacrylate copolymer, polyether, shellac, or combinations thereof. In some embodiments, the pH-dependent polymer coat comprises methacrylic acid-methyl methacrylate copolymer.

The pH-independent polymer coat or the pH-dependent polymer coat may further include a pharmaceutically acceptable plasticizer. The plasticizer may include triethyl citrate (TEC), triacetin, acetyl tributyl citrate, acetyl triethyl citrate, glycerin, a polyethylene glycol, polyethylene glycol monomethyl ether, propylene glycol, sorbitol sorbitan solution, castor oil, diacetylated monoglycerides, dibutyl sebacates, diethyl phthalate or combinations thereof. In some embodiments, the plasticizer comprises triethyl citrate. In some embodiments, the pH-independent polymer coat or the pH-dependent polymer coat is present on the sustained release bead at a concentration of 15.0 wt %-50.0 wt % of the weight of the sustained release bead. The pH-independent polymer coat or the pH-dependent polymer coat may be present on the sustained release bead at a concentration of 20.0 wt %-40.0 wt % of the weight of the sustained release bead.

In some embodiments, the dosage form disclosed herein, comprises a total of 6 mg-72 mg of deutetrabenazine. In some embodiments, the dosage form comprises a total of 6 mg, or 12 mg, or 18 mg, or 24 mg, or 30 mg, or 36 mg, or 42 mg or 48 mg of deutetrabenazine.

The dosage form disclosed herein may consist essentially of a population of sustained release beads comprising a pH-independent polymer coat or a population of sustained release beads comprising a pH-independent polymer coat further coated with a pH-dependent polymer coat. The dosage form may be a capsule, a sachet or the like.

In some embodiments, the dosage form consists essentially of a population of sustained release beads comprising
a) a core comprising nanonized deutetrabenazine and the pharmaceutically acceptable excipient; wherein the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate and sodium bicarbonate, and a surfactant comprising sodium lauryl sulfate;
b) a pH-independent polymer coat coating the core; and optionally further comprising
c) a capsule shell or pharmaceutical sachet packaging.

The core may be in the form of immediate release granules, immediate release pellet or immediate release tablet or an inert particle coated with deutetrabenazine and the pharmaceutically acceptable excipient. In some embodiments, the pH-independent polymer coat comprises ethylcellulose. In some embodiments, the pH-independent polymer coat comprises ethylcellulose, polyethylene glycol and triethyl citrate, and optionally further comprises povidone. In some embodiments, the pH-independent polymer coat comprises a mixture of cellulose acetate NF 398-10 and cellulose acetate 320S. In some embodiments, the pH-independent polymer coat comprises cellulose acetate and optionally polyethylene glycol.

In various embodiments, the dosage form comprises a population of sustained release beads and further comprises a population of immediate release beads. The immediate release beads comprise one of a) immediate release granules, immediate release pellet or immediate release tablet comprising an amount of deutetrabenazine and a pharmaceutically acceptable excipient or b) an inert particle coated with an amount of deutetrabenazine and a pharmaceutically acceptable excipient. In some embodiments, the immediate release beads include (b).

In some embodiments, the dosage form comprises a population of immediate release beads and a population of sustained release beads, the sustained release beads comprising
a) a core comprising the deutetrabenazine and the pharmaceutically acceptable excipient; wherein the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate and sodium bicarbonate, and a surfactant comprising sodium lauryl sulfate;
b) a pH-dependent polymer coat sensitive to pH 5.5-pH 7 coating the core.
   The pH-dependent polymer coat may include methacrylic acid-ethyl acrylate copolymer, hydroxypropylmethyl cellulose phthalate (HPMCP), alginates, carboxymethylcellulose, or a combination thereof. Without wishing to be bound to any particular theory, the pH-dependent polymer coat comprising methacrylic acid and ethyl acrylate copolymer, and triethyl citrate is sensitive in a pH of about 5.5 to about 7, thereby targeting the small intestine.

In some embodiments, the dosage form comprises a population of sustained release beads comprising
a) a core comprising the deutetrabenazine and the pharmaceutically acceptable excipient; wherein the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate and sodium bicarbonate, and a surfactant comprising sodium lauryl sulfate;
b) a pH-dependent polymer coat sensitive to a pH 7 to about pH 8 coating the core.

The pH-dependent polymer coat sensitive to pH >7.0 may be cellulose acetate phthalate, hydroxypropyl methylcellulose phthalate, hydroxypropyl methylcellulose succinate, polyvinyl acetate phthalate, pH-sensitive methacrylic acid-methyl methacrylate copolymer, polyether, shellac, or combinations thereof. Without wishing to be bound to any particular theory, the pH-dependent polymer coat comprising methacrylic acid and methyl acrylate copolymer, and triethyl citrate is sensitive to a pH of about 7 to about 8, thereby dissolving in the large intestine/colon.

The core of the aforementioned sustained release beads comprises a) immediate release granules, immediate release pellet or immediate release tablet comprising deutetrabenazine and the pharmaceutically acceptable excipient or b) an inert particle coated with deutetrabenazine and the pharmaceutically acceptable excipient. In some embodiments, the core comprises (b).

In some embodiments, the dosage form disclosed herein includes a population of immediate release beads and a population of sustained release beads, the sustained release beads having a pH-dependent coating that dissolves at pH 5.5-7.

In some embodiments, the dosage form disclosed herein includes a population of immediate release beads and a population of sustained release beads, the sustained release beads having a pH-dependent coating that dissolves at pH >7.

In some embodiments, the dosage form disclosed herein includes a population of immediate release beads and two populations of sustained release beads, one population of the sustained release beads having a pH-dependent coating that dissolves at pH 5.5-7.0, and a second population of the sustained release beads having a pH-dependent coating that dissolves at pH >7.

The dosage forms disclosed herein may be in the form of a capsule, comprising a capsule shell and at least one population of sustained release beads, optionally further comprising a population of immediate release beads. Alternatively, the dosage forms disclosed herein may be in the form of a sachet, comprising a sachet package and at least one population of sustained release beads, optionally further comprising a population of immediate release beads

In some embodiments, about 10 wt %-30 wt % of deutetrabenazine is released from the dosage form within 1 hour, and about 50-80 wt % of deutetrabenazine is released within 3 hours and not less than (NLT) about 80 wt % of deutetrabenazine is released within 5 hours as measured in a USPIII dissolution device, pH 7.2.

In other embodiments, the dosage forms of the disclosure release about 40-60 wt % of deutetrabenazine within 7 hours, as measured in a USPII device, pH3.0 phthalate buffer, 75 rpm. Further provided herein, are methods useful in treating VMAT2 mediated disorders. In some embodiments, the method of treating a VMAT2 mediated disorder comprises orally administering to a patient in a need thereof, the controlled release dosage form disclosed herein. The VMAT2 mediated disorder may be a hyperkinetic movement disorder. The hyperkinetic movement disorder may be a chronic disorder, for example Huntington's disease, tardive dyskinesia, and dyskinesia in cerebral palsy.

Further provided herein is a process for manufacturing the immediate release beads or the core of the sustained release beads, comprising the steps of
a) providing a dispersion of nanonized deutetrabenazine with a pharmaceutically acceptable excipient, wherein the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate and sodium bicarbonate, and a surfactant comprising sodium lauryl sulfate;
b) forming immediate release granules, immediate release pellet or immediate release tablet from the dispersion of a); or coating an inert particle with the dispersion of a); thereby generating the immediate release beads or the core of the sustained release beads.

Further provided is a process for manufacturing the sustained release beads comprising the steps of
a) providing a deutetrabenazine dispersion comprising nanonized deutetrabenazine and a pharmaceutically acceptable excipient, wherein the pharmaceutically acceptable excipient comprises an antioxidant, a water-soluble binder c, an anti-foaming agent, a filler, and a surfactant;
b) providing a core, wherein the core comprises immediate release granules, immediate release pellet or immediate release tablet comprising the dispersion of a); or an inert particle coated with the dispersion of a);
c) coating the core of b) with a pH-independent polymer coating, a pH-dependent polymer coating or with a pH-independent polymer coating and a pH-dependent polymer coating;
thereby generating sustained release beads.

In some embodiments, the nanonized deutetrabenazine is prepared by milling. In some embodiments the pharmaceutically acceptable excipient consists of an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate and sodium bicarbonate, and a surfactant comprising sodium lauryl sulfate. Further provided is nanonized deutetrabenazine having a median particle size or about 0.02 to about 2.0 micron. In some embodiments, the nanonized deutetrabenazine has a particle size distribution characterized by a D90 of about 0.8 to 1.6 micron, a D50 of about 0.2 to about 0.6 micron and a D10 of about 0.1 to about 0.2 micron.

### BRIEF DESCRIPTION OF THE DRAWINGS

FIGS. 1A-1D provide illustrations of bead populations. Figure TA shows three options for the core of sustained release beads or the immediate release beads. The left most figure represents granules, pellet or tablet comprising deutetrabenazine and excipient; the left figure represents granules, pellet or tablet comprising deutetrabenazine and further optionally coated with deutetrabenazine dispersion; and the right most figure represent a deutetrabenazine dispersion coated inert particle. FIGS. 1B, 1C and 1D show possible sustained release beads, based on the core/immediate release beads in Figure TA. FIG. 1B shows populations of sustained release beads which cores coated with a pH-independent polymer (black layer). FIG. 1C shows populations of sustained release beads which are cores coated with a pH-dependent polymer (sensitive to pH5.5-pH7; dotted layer) or coated with a pH-dependent layer (black layer) and further coated with a pH-dependent polymer (sensitive to pH5.5-pH7; dotted layer). FIG. 1D shows populations sustained release beads which are cores coated with a pH-dependent polymer (sensitive to pH>7; striped layer) or coated with a pH-dependent layer (black layer) and further coated with a pH-dependent polymer (sensitive to pH>7; striped layer).

FIG. 2 provides a flowchart exemplifying the general manufacturing process for a deuterated dispersion coated inert particle. The particle may serve as an immediate release bead or as a core for a sustained release bead.

FIG. 3 shows the dissolution profiles of Samples 1, 2, and 3 in 500 mL Phosphate Buffer pH 6.8, USPII apparatus, 75 rpm. The micro-milled and nano-milled particles exhibit a better dissolution profile in pH 6.8 than un-milled sample. The diamonds represent release profile of the unmilled sample, showing poor release (˜30-35 wt % even after 2 hr); the squares represent the release profile of the micro-milled deutetrabenazine and the triangles represent the release profile of the nano-milled deutetrabenazine.

FIG. 4 is a graph showing dissolution of a dosage form comprising immediate release beads, and two populations of sustained release beads, one population with a coating sensitive to pH5.5-7.0 and the second population with a coating sensitive to pH. 7.0. Dissolution was performed in 250 mL 0.1N HCl (1 hr), pH 6.8 Phosphate Buffer (2 hrs), pH 7.2 Phosphate Buffer (3 hrs) using USPIII, 10 dpm. The units of the x axis are in hours.

FIG. 5 is a graph showing the dissolution profiles for Samples 5-10 in pH3.0 phthalate buffer, USPII, 75 rpm.

FIG. 6 is a graph showing the dissolution profiles for Samples 11-12 in pH3.0 phthalate buffer, USPII, 75 rpm.

FIG. 7 is a graph showing the dissolution profiles for Samples 13-16 in pH3.0 phthalate buffer, USPII, 75 rpm.

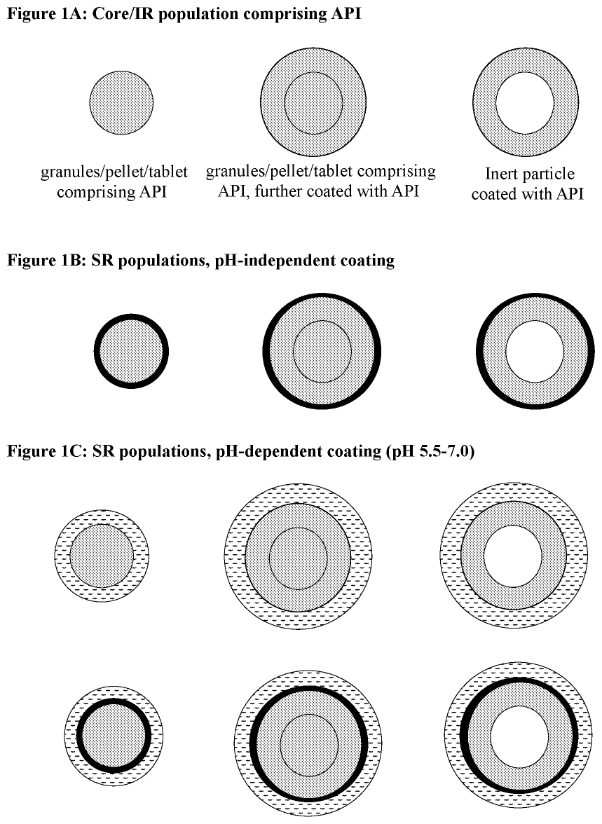

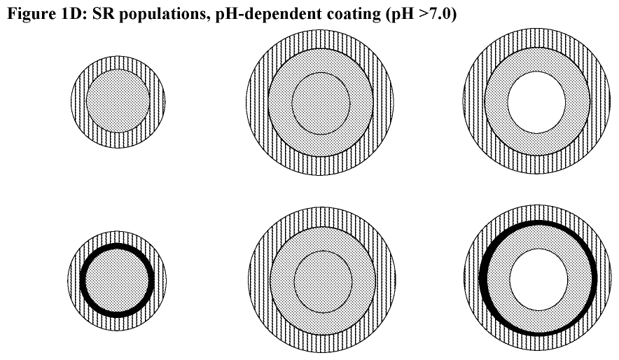

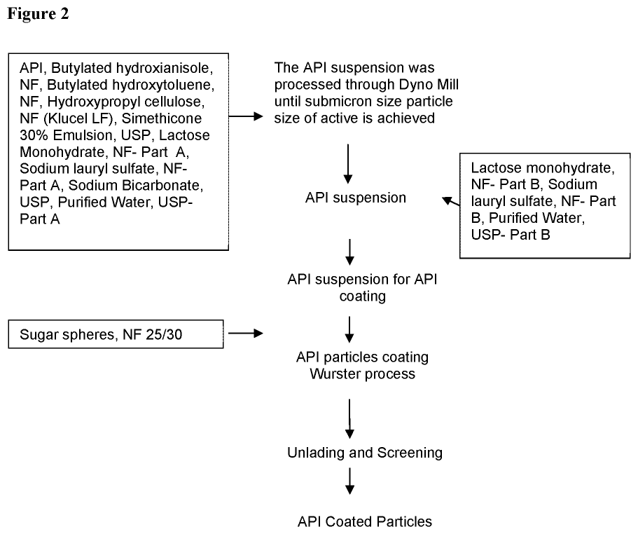

### DETAILED DESCRIPTION OF THE INVENTION

The present subject matter may be understood more readily by reference to the following detailed description, which forms a part of this disclosure. It is to be understood that this invention is not limited to the specific methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art.

As employed above and throughout the disclosure, the following terms and abbreviations, unless otherwise indicated, shall be understood to have the following meanings.

The singular forms “a,” “an,” and “the” may refer to plural articles unless specifically stated otherwise.

The term “about,” as used herein, is intended to qualify the numerical values which it modifies, denoting such a value as variable within a margin of +10%.

When a range of values is expressed, another embodiment includes from the one particular and/or to the other particular value. Similarly, when values are expressed as approximations, by use of the antecedent “about,” it is understood that the particular value forms another embodiment. All ranges are inclusive and combinable.

As used herein, the terms “compound”, “drug”, “pharmacologically active agent”, “active agent”, or “medicament” are used interchangeably herein to refer to a compound or compounds or composition of matter which, when administered to a subject (human or animal) induces a desired pharmacological and/or physiologic effect by local and/or systemic action. The active agent disclosed herein is preferably deutetrabenazine. “Deutetrabenazine” or “deu-TBZ” is a selectively deuterium-substituted, stable, non-radioactive isotopic form of tetrabenazine in which the six hydrogen atoms on the two O-linked methyl groups have been replaced with deuterium atoms (i.e. —OCD3 rather than —OCH3 moieties).

As used herein, “dosage form” refers to a drug form having multiparticulate properties wherein each bead population exhibits different properties. In some embodiments, the dosage form is made up of a single population of sustained release beads. In some embodiments, the dosage form is made up of more than one population of sustained release beads. In some embodiments, the dosage form is made up of at least one population of sustained release beads and at least one population of immediate release beads.

The term “bead” as used herein refers to a discrete unit of the pharmaceutical formulation comprising at least deutetrabenazine and a pharmaceutically acceptable excipient. In some embodiments, an immediate release bead refers to an immediate release formulation comprising a core, which can be formed from granules, a pellet or a tablet comprising the deutetrabenazine and a pharmaceutically acceptable excipient. In some embodiments, the immediate release bead comprises the core, e.g. granules, pellet, tablet further at least partially coated with deutetrabenazine and a pharmaceutically acceptable excipient. In other embodiments, the immediate release bead comprises an inert particle, such a microcrystalline cellulose (MCC) or sugar particle, at least partially coated with deutetrabenazine and a pharmaceutically acceptable excipient. The sustained release beads disclosed herein, comprise an immediate release core or immediate release particle (i.e. deutetrabenazine containing granules, pellet, tablet, coated inert particle) that is further coated with a pH-independent polymer and/or pH-dependent polymer.

The term “immediate release” (IR) as used herein refers to a pharmaceutical formulation, i.e. bead, which releases the active agent, i.e. deutetrabenazine, within about one hour post administration. Such release typically occurs in the upper gastrointestinal (GI) tract, for example in the stomach.

The term “sustained release” as used herein refers to a pharmaceutical formulation, i.e. bead, which releases the active agent, i.e. deutetrabenazine, over a prolonged period of time, typically from 1 to 12, or from 1 to 24 hr post administration. Such release typically occurs in the gastrointestinal (GI) tract for example in the upper intestine and/or lower intestines and/or colon.

“Controlled release” refers to a dosage form able to release active agent over an extended period for example, up to about 7 hours, up to about 12 hours, up to about 15 hours, up to about 18 hours, up to about 21 hours or up to about 24 hours. The active agent is preferably deutetrabenazine, as disclosed herein. Some of the active agent is released in the stomach (immediate release) and some in the small intestine and/or lower intestine/colon (sustained release). In some embodiments, the dosage form releases about 10 wt %-30 wt % of the active agent in the dosage form within one hour; about 50 wt %-80 wt % within 3 hours and not less than 80% after 5 hours as measured in a USPIII apparatus, pH7.2. In other embodiments, the dosage form releases about 5-40-60 wt % of the active agent in the dosage form within 7 hours, as measured in a USPII device, pH3.0 phthalate buffer, 75 rpm. In other embodiments, the dosage form releases about 50 wt % of deutetrabenazine within 7 hours, as measured in a USPII device, pH3.0 phthalate buffer, 75 rpm.

The dosage forms as disclosed herein can be in the form of a capsule or otherwise packaged beads. A “capsule” is a dosage form encasing the bead populations, as disclosed herein. The capsule may be formed of gelatin (animal or vegetable) or other pharmaceutically acceptable material.

The gastrointestinal tract or “GI tract”, which extends from the oral cavity, through the esophagus, to the stomach and though the small intestine and colon to the anus, exhibits differing pH depending on the region and food status. The stomach is typically the first section of the GI tract in which disintegration and dissolution of drugs take place. The pH of the stomach is normally 1-3. The intestines are the main absorption site for nutrients and drugs. The small intestine has three distinct regions, duodenum, jejunum, and ileum. The entry of solid dosage forms into the small intestine is accompanied by a sharp pH increase because of the duodenal secretion of bicarbonate. Moreover, the literature suggests a subsequent increase of the pH value from about pH 5.5-6.8 in the duodenum to pH 6.8-8 in the terminal ileum. The pH values in the large intestine (including the colon), are slightly more acidic compared with the ileal pH values possibly due to the fermentation processes of the colonic microbiota (Koziolek, et al, J Pharma Sci; 104(9) 2855-63).

As used herein, the terms “method of treatment” or “therapy” (as well as different forms thereof) include preventative (e.g., prophylactic), curative, or palliative treatment. As used herein, the term “treating” includes alleviating or reducing at least one adverse or negative effect or symptom of a condition, disease or disorder. This condition, disease or disorder may refer to hyperkinetic movement disorder, such as, but not limited to, Huntington's disease, tardive dyskinesia, Tourette syndrome, dystonia, dyskinesia in cerebral palsy (DCP) and levodopa-induced dyskinesia (LID in Parkinson's disease.

The term “administering” means providing to a patient the pharmaceutical composition or dosage form (used interchangeably herein) of the present invention.

The terms “subject”, “individual”, and “patient” are used interchangeably herein, and refer to a human, to whom treatment, including prophylactic treatment, with the dosage form according to the present invention, is provided.

“Pharmaceutically acceptable” refers to those compounds, materials, compositions, and/or excipients which are, within the scope of sound medical judgment, suitable for contact with the tissues of human beings without excessive toxicity, irritation, allergic response, or other problem complications commensurate with a reasonable benefit/risk ratio.

The terms D90, D50 or D10 are well understood in the art. For example, a D90 of 15 μm, means that 90% (by volume) of the particles have a size less than or equal to 15 μm. A D50 of 10 μm, means that 50% (by volume) of the particles have a size less than or equal to 10 μm. A D10 of 3 μm, means that 10% (by volume) of the particles have a size less than or equal to 3 μm. The terms may be combined to define a particle size distribution (PSD).

Particle size distribution is determined by means of laser diffractometry. More specifically, the particle size distribution was determined using a Mastersizer 3000 from Malvern Instruments. The particle size determination may be carried out as a wet or dry measurement depending on the sample.

Although constant-release dosage forms have been proven effective for many different drug therapies, there are clinical situations where these have not been entirely satisfactory. It has been observed that for some patients, the therapeutic effectiveness of the drug decreases below the therapeutically effective threshold before the end of the desired therapy period despite the maintenance of substantially constant drug release that would be expected to provide continued effectiveness.

It has been surprisingly discovered that oral dosage forms comprising deutetrabenazine that exhibit a desirable rate of release and hence a desirable pharmacokinetic profile for an extended time can be achieved. In some embodiments, the presently disclosed multiparticulate dosage forms when administered orally to a subject on a once daily basis (qd) provide a pharmacokinetic profile that is comparable, e.g., bioequivalent, to that of the AUSTEDO® dosage forms administered twice daily (bid).

Provided herein is a controlled release oral dosage form for once daily administration of deutetrabenazine comprising a population of sustained release beads; wherein the sustained release beads comprise a core comprising deutetrabenazine and a pharmaceutically acceptable excipient; and further comprising a pH-independent polymer coat, a pH-dependent polymer coat or a pH-independent polymer coat further coated with a pH-dependent polymer coat. In some embodiments, the core comprises immediate release granules, immediate release pellet or immediate release tablet that comprises deutetrabenazine and a pharmaceutically acceptable excipient. The deutetrabenazine and pharmaceutically acceptable excipient may be a deutetrabenazine dispersion. In some embodiments, the core comprises an inert particle for example, a crystalline microcellulose particle or a sugar particle. Such particles are well known to the formulator skilled in the art. In such embodiments, the core comprises an inert particle coated with the deutetrabenazine dispersion.

In some embodiments, the dosage form further comprises a population of immediate release beads; wherein the population of immediate release beads comprises a) immediate release granules, immediate release pellet or immediate release tablet comprising an amount of deutetrabenazine and a pharmaceutically acceptable excipient or b) an inert particle coated with an amount of deutetrabenazine and a pharmaceutically acceptable excipient. In some embodiments, a portion of the immediate release granules, pellet or tablet or inert particle of the immediate release beads serves as the core of the sustained release beads.

It is now related that the dosage form performs as disclosed when the deutetrabenazine has a median particle size of 0.02 to 2.0 micron (μm), or 0.2 to 1.6 micron, or 0.15 to 1.2 micron, 0.15 to 1.0 micron, 0.5 to 1.6 micron or about 0.8 to about 1.6 micron. The desired median particle size may be generated by, for example, milling the drug substance to low micrometer and nanometer sizes. In some embodiments, the deutetrabenazine has a particle size distribution characterized by a D₉₀ of about 0.8 to about 1.6 micron. The D₉₀ is preferably about 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, 1.00, 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.21, 1.22, 1.23, 1.24, 1.25, 1.26, 1.27, 1.28, 1.29, 1.30, 1.31, 1.32, 1.33, 1.34, 1.35, 1.36, 1.37, 1.38, 1.39, 1.40, 1.41, 1.42, 1.43, 1.44, 1.45, 1.46, 1.47, 1.48, 1.49, 1.50, 1.51, 1.52, 1.53, 1.54, 1.55, 1.56, 1.57, 1.58, 1.59 or about 1.60 μm. In some embodiments, the deutetrabenazine has a particle size distribution characterized by a D₅₀ of about 0.1 to about 0.6 micron, or about 0.2 to about 0.6 micron. The D₅₀ is preferably about 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59 or about 0.60 μm. In some embodiments, the deutetrabenazine has a particle size distribution characterized by a D₁₀ of about 0.1 to about 0.2 micron. The D₁₀ is preferably about 0.10, 0.11, 0.12, 0.13, 0.14, 0.15, 0.16, 0.17, 0.18, 0.19, or about 0.20 μm. In some embodiments, the deutetrabenazine has a particle size distribution characterized by a D₉₀ of about 0.8 to 1.6 micron, a D50 of about 0.2 to about 0.6 micron and a D10 of about 0.1 to about 0.2 micron.

In some embodiments, the deutetrabenazine is present in the core or immediate release bead in a range of about 5 wt %-80 wt %, or 10 wt %-80 wt %, or 10 wt %-70 wt %, 20 wt %-60 wt %, 5 wt %-30 wt %, or 50 wt %-80 wt % of total weight of the dosage form. Deutetrabenazine may be present in the core or immediate release bead in an amount of about (by wt %) 5.0, 6.0, 7.0, 8.0, 9.0, 10.0, 1.01, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, 50.0, 60.0, 61.0, 62.0, 63.0, 64.0, 65.0, 66.0, 67.0, 68.0, 69.0, 70.0, 71.0, 72.0, 73.0, 74.0, 75.0, 76.0, 77.0, 78.0, 79.0, 70.0, wt % of the weight of the core or in the immediate release bead, respectively.

The pharmaceutically acceptable excipient comprises an antioxidant, a binder, a filler, a surfactant, an anti-foaming agent or combinations thereof. Typically, more than one excipient is used. In some embodiments, the excipient comprises an antioxidant, which is a water-insoluble antioxidant. In some embodiments, the water-insoluble antioxidant is selected from the group consisting of propyl gallate, 6-ethoxy-1,2-digydro-2,2,4-trimethylquinoline (ethoxyquin), nordihydroguaiaretic acid (NDGA), butylated hydroxyanisole, butylated hydroxytoluene or any mixture thereof. In one specific embodiment, the antioxidant is selected from butylated hydroxytoluene (BHT), butylated hydroxyanisole (BHA), and combinations thereof. The antioxidant, preferably the water-insoluble antioxidant, is present in the dosage form in a range of 0.1 wt %-1.0 wt %, or about 0.2 wt %-1.0 wt %, or about 0.5 wt %-0.8 wt % of the weight of the core or in the immediate release bead and may be present in an amount of (by wt %) 0.10, 0.11, 0.12, 0.13, 0.14, 0.15, 0.16, 0.17, 0.18, 0.19, 0.20, 0.21, 0.22, 0.23, 0.24, 0.25, 0.26, 0.27, 0.28, 0.29, 0.30, 0.31, 0.32, 0.33, 0.34, 0.35, 0.36, 0.37, 0.38, 0.39, 0.40, 0.41, 0.42, 0.43, 0.44, 0.45, 0.46, 0.47, 0.48, 0.49, 0.50, 0.51, 0.52, 0.53, 0.54, 0.55, 0.56, 0.57, 0.58, 0.59, 0.60, 0.61, 0.62, 0.63, 0.64, 0.65, 0.66, 0.67, 0.68, 0.69, 0.70, 0.71, 0.72, 0.73, 0.74, 0.75, 0.76, 0.77, 0.78, 0.79, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, 0.99, or 1.0 wt % of the weight of the core or in the immediate release bead, respectively.

The excipient may comprise a binder. In some embodiments, the binder comprises a water-soluble binder, a water-insoluble binder or combinations thereof. In some embodiments, the binder comprises a water-soluble binder which may be a cellulose based binder including hydroxypropyl cellulose, and hydroxypropyl methylcellulose, polyvinyl pyrrolidone, polyvinyl alcohol, a polyacrylic acid polymer, polyether, carbohydrate polymer (natural or synthetic) or combinations thereof. In some embodiments, the binder is a cellulose-based binder selected from the group consisting of methyl cellulose (MC), ethyl cellulose (EC), propyl cellulose (PC), hydroxymethyl cellulose (HMC), hydroxyethyl cellulose (HEC), hydroxypropyl cellulose (HPC), hydroxypropyl methyl cellulose (HPMC), cellulose acetate and combinations thereof. In some embodiments, the binder is hydroxypropyl cellulose. In some embodiments, the binder is a polyether. Suitable polyethers include polyethylene glycol polymers. In further embodiments, the binder comprises a water-insoluble polymer, which comprises crospovidone, copovidone, microcrystalline cellulose, croscarmellose sodium, starch, sodium starch glycolate, colloidal silica, silica, ethyl cellulose, lactic acid polymer, a lactic acid and glutamic acid copolymer, polyvinyl acetate or combinations thereof. In some embodiments, the binder is present in the core or in the immediate release bead in a range of 0.5 wt %-10.0 wt %, about 1.0 wt %-8.0 wt %, or about 2.0 wt %-6.0 wt % of the weight of the dosage form. The binder may present in the dosage form in an amount of (by wt. %) 0.5, 1.0, 1.5, 2.0, 2.5, 3.0, 3.5, 4.0, 4.5, 5.0, 5.5, 6.0, 6.5, 7.0, 7.5, 8.0=, 8.5, 9.0, 9.5, or about 10.0 wt % of the weight of the core or in the immediate release bead, respectively.

In some embodiments, the excipient comprises a filler selected from the group consisting of a saccharide, a disaccharide, a polysaccharide, a polyalcohol, microcrystalline cellulose, natural and synthetic gums, gelatin, pregelatinized starch, polyvinylpyrrolidone, cellulose derivatives, dibasic calcium phosphate, kaolin, inorganic salts, calcium carbonate, sodium bicarbonate, sodium carbonate and combinations thereof. The saccharide may be for example, glucose, galactose, dextrose, fructose; a disaccharide may be for example, sucrose, lactose, lactose monohydrate, maltose, trehalose, maltose; a polysaccharide may be starch, maltodextrin; and a polyalcohol may be for example, sorbitol, xylitol, inositol, lactitol, mannitol, spray-dried mannitol. In some embodiments, the filler is microcrystalline cellulose, lactose monohydrate or a combination thereof. In some embodiments, the filler is present in the dosage form in a range of 5.0-50.0 wt %, 5.0-30.0 wt %, 10.0-40.0 wt %, or 10.0-40.0 wt %, of the weight of the core or in the immediate release bead. In some embodiments, the excipient comprises about (by wt %) 5.0, 6.0, 7.0, 8.0, 9.0, 10.0, 1.01, 12.0, 13.0, 14.0, 15.0, 16.0, 17.0, 18.0, 19.0, 20.0, 21.0, 22.0, 23.0, 24.0, 25.0, 26.0, 27.0, 28.0, 29.0, 30.0, 31.0, 32.0, 33.0, 34.0, 35.0, 36.0, 37.0, 38.0, 39.0, 40.0, 41.0, 42.0, 43.0, 44.0, 45.0, 46.0, 47.0, 48.0, 49.0, or 50 wt % of the weight of the core or in the immediate release bead, respectively.

In some embodiments of the dosage form, the pharmaceutically acceptable excipient comprises a surfactant. The surfactant may comprises sodium lauryl sulfate, sodium dodecyl sulfate, sodium laureth sulfate, docusate sodium, polysorbate, tween, polyoxyethylene 15 hydroxy stearate, polyoxyethylene castor oil derivatives, polyoxyethylene stearates, sorbitan fatty acid esters, polyoxyethylene alkyl ethers, polyoxyethylene nonylphenol ether or combinations thereof. In some embodiments, the surfactant is present in the core or in the immediate release bead at a concentration of 2.0 wt %-12.0 wt % of the weight of the core or the immediate release bead. The surfactant may present in the core or in the immediate release bead in an amount of (by wt %), 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0. or 12.0 wt % of the weight of the core or in the immediate release bead, respectively.

In some embodiments, the excipient comprises an anti-foaming agent, for example insoluble oils, polydimethylsiloxanes and other silicones, certain alcohols, stearates, glycols and combinations thereof. In various embodiments, the anti-foaming agent is simethicone, dimethicone, tilactase or peppermint oil. The anti-foaming agent may be simethicone 30% at up to about 2.0 wt % of the weight of the core or in the immediate release bead.

In some embodiments, an immediate release bead disclosed herein comprises an inert particle coated with nanonized deutetrabenazine having a median particle size of 0.02 to 2.0 micron (μm) and a pharmaceutically acceptable excipient comprising about 0.1 wt %-1.0 wt % an antioxidant, about 0.5 wt %-10.0 wt % of a binder, about of 5.0 wt %-50.0 wt % of a filler, about 2.0 wt %-12.0 wt % of a surfactant, and about 0.3 wt %-3 wt % an anti-foaming agent, of the weight core or in the immediate release bead.

In some embodiments, the sustained release beads comprise a pH-independent polymer coat coating the core. The pH-independent polymer coat may include ethylcellulose. In some embodiments, the pH-independent polymer coat includes a cellulose acetate, a mixture of cellulose acetates, ethylcellulose or a mixture of ethylcellulose and polyethylene glycol. In some embodiments, the pH-independent polymer coat comprises cellulose acetate. In some embodiments, the pH-independent polymer coat comprises a mixture of cellulose acetate NF 398-10 and cellulose acetate 320S. In yet other embodiments, the pH-independent polymer coat comprises a mixture of cellulose acetate and polyethylene glycol.

In certain embodiments, the sustained release beads comprise a pH-dependent polymer coat surrounding the core. In some embodiments, the sustained release bead is coated with a pH-dependent polymer to target drug release at a pH 5-7.0 and targets the upper small intestine. The enteric polymer is methacrylic acid-ethyl acrylate copolymer. In some embodiments the sustained release bead is coated with a pH-dependent polymer to target drug release at a pH >7.0 and targets the large intestine/colon. In some embodiments, the pH-dependent polymer coat that targets large intestine/colon comprises cellulose acetate phthalate, hydroxypropyl methylcellulose phthalate, hydroxypropyl methylcellulose succinate, polyvinyl acetate phthalate, pH-sensitive methacrylic acid-methyl methacrylate copolymer, polyether, shellac and combinations thereof. In some embodiments, the pH-dependent polymer coat comprises methacrylic acid-methyl methacrylate copolymer. In some embodiments, the pH-dependent polymer coat comprises a mixture of cellulose acetate and polyethylene glycol. In some embodiments, the pH-dependent polymer coat comprises a mixture of ethyl cellulose and polyethylene glycol.

The pH-independent or pH-dependent polymer coat may further include a pharmaceutically acceptable plasticizer. The plasticizer may be triethyl citrate (TEC), triacetin, acetyl tributyl citrate, acetyl triethyl citrate, glycerin, a polyethylene glycol, polyethylene glycol monomethyl ether, propylene glycol, sorbitol sorbitan solution, castor oil, diacetylated monoglycerides, dibutyl sebacates, diethyl phthalate or combinations thereof. In some embodiments, the plasticizer comprises triethyl citrate.

In some embodiments of the dosage form, the pH-independent polymer coat or the pH-dependent polymer coat is present on the sustained release bead at a concentration of 15.0 wt %-50.0 wt %, or about 20.0 wt %-40.0 wt % of the weight of the sustained release bead.

The dosage form may include a total of 6 mg-72 mg of deutetrabenazine. In some embodiments, the dosage form comprises a total of 6 mg, or 12 mg, or 18 mg, or 24 mg, or 30 mg, or 36 mg, or 42 mg or 48 mg of deutetrabenazine.

In some embodiments, the dosage form consists essentially of a population of sustained release beads comprising
a) a core comprising deutetrabenazine and a pharmaceutically acceptable excipient; wherein the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate and sodium bicarbonate, and a surfactant comprising sodium lauryl sulfate;
b) a pH-independent polymer coat coating the core; and optionally further comprising
c) a capsule shell or pharmaceutical sachet packaging.

The core of the dosage form comprises a) immediate release granules, immediate release pellet or immediate release tablet comprising the deutetrabenazine and the pharmaceutically acceptable excipient or b) an inert particle coated with the deutetrabenazine and the pharmaceutically acceptable excipient.

In some embodiments, the pH-independent polymer comprises ethylcellulose, polyethylene glycol and triacetin, optionally further comprising povidone. In other embodiments, the pH-independent polymer coat comprises cellulose acetate and optionally polyethylene glycol (PEG). In some embodiments, the cellulose acetate comprises a mixture of cellulose acetate 398-10 and cellulose acetate 320S, optionally further comprising PEG 3350.

In some embodiments, the dosage form comprises at least one population of sustained release beads and one population of immediate release beads, wherein the immediate release beads comprise a) immediate release granules, immediate release pellet or immediate release tablet comprising the deutetrabenazine and the pharmaceutically acceptable excipient or b) an inert particle coated with the deutetrabenazine and the pharmaceutically acceptable excipient. In some embodiments, the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate and sodium bicarbonate, and a surfactant comprising sodium lauryl sulfate. The sustained release beads comprise a core, which may consist essentially of the immediate release beads, further comprising a pH-dependent polymer coat that targets the small intestine. In some embodiments, the pH-dependent polymer coat comprises methacrylic acid and ethyl acrylate copolymer, and optionally triethyl citrate. In other embodiments, the sustained release beads comprise a core, which may consist essentially of the immediate release beads, further comprising a pH-dependent polymer coat that targets the large intestine/colon. In some embodiments, the pH-dependent polymer coat comprises methacrylic acid and methyl acrylate copolymer, and optionally triethyl citrate.

In some embodiments, the dosage form comprises two populations of sustained release beads and one population of immediate release beads, one population of the sustained release beads targeting the small intestine and a second population of the sustained release beads targeting the large intestine/colon. The dosage form may be, for example, a capsule or a pharmaceutical sachet package.

Further provided herein, are methods useful in treating VMAT2 mediated disorders. In some embodiments, the method of treating a VMAT2 mediated disorder comprises orally administering to a patient in a need thereof, the controlled release dosage form disclosed herein. The VMAT2 mediated disorder may be a hyperkinetic movement disorder. The hyperkinetic movement disorder may be a chronic disorder, for example dystonia, dyskinesia, Huntington's disease, tardive dyskinesia, and dyskinesia in cerebral palsy. In some embodiments, the method is effective in treating chorea associated with Huntington's disease. In some embodiments, the method is effective in treating tardive dyskinesia. The subjects afflicted with tardive dyskinesia may be concurrently administered an antipsychotic agent. In some embodiments, the method is effective in treating dyskinesia in cerebral palsy.

In certain embodiments, the multiparticulate dosage form according to any one of the embodiments disclosed herein, is administered with food.

In certain embodiments, the multiparticulate dosage form according to any one of the embodiments disclosed herein, is administered under fasting conditions.

The plasma profiles of the dosage form following administration are favorable. In one embodiment, a single dose administration of the oral dosage form comprising 6 mg of deutetrabenazine provides an in vivo plasma profile for total α- and β-dihydrodeutetrabenazine that includes a geometric mean AUC0-inf of about 90,000 to 142,750 h*pg/mL and/or a geometric mean Cmax of less than about 4,600 pg/mL.

In one embodiment, a single dose administration of the oral dosage form comprising 12 mg of deutetrabenazine provides an in vivo plasma profile for total α- and β-dihydrodeutetrabenazine that includes a geometric mean AUC0-inf of about 180,000 to 285,500 h*pg/mL and/or a geometric mean Cmax of less than about 9,200 pg/mL.

In one embodiment, a single dose administration of the oral dosage form comprising 24 mg of deutetrabenazine provides an in vivo plasma profile for total α- and β-dihydrodeutetrabenazine that includes a geometric mean AUC0-inf of about 360,000 to 571,000 h*pg/mL and/or a geometric mean Cmax of less than about 18,400 pg/mL.

In one embodiment, a single dose administration of the oral dosage form comprising 36 mg of deutetrabenazine provides an in vivo plasma profile for total α- and β-dihydrodeutetrabenazine that includes a geometric mean AUC0-inf of about 540,000 to 856,500 h*pg/mL and/or a geometric mean Cmax of less than about 27,600 pg/mL.

In one embodiment, a single dose administration of the oral dosage form comprising 48 mg of deutetrabenazine provides an in vivo plasma profile for total α- and β-dihydrodeutetrabenazine that includes a geometric mean AUC0-inf of about 720,000 to 1,142,000 h*pg/mL and/or a geometric mean Cmax of less than about 36,800 pg/mL.

In one embodiment, administration of the oral dosage form comprising 6 mg of deutetrabenazine provides an in vivo plasma profile for total α- and β-dihydrodeutetrabenazine that includes a geometric mean AUC0-24 of about 102,500 to 200,000 h*pg/mL at steady state and/or a mean Cmax of less than about 10,000 pg/mL at steady state.

In one embodiment, administration of the oral dosage form comprising 12 mg of deutetrabenazine provides an in vivo plasma profile for total α- and β-dihydrodeutetrabenazine that includes a geometric mean AUC0-24 of about 205,000 to 400,000 h*pg/mL at steady state and/or a mean Cmax of less than about 20,000 pg/mL at steady state.

In one embodiment, administration of the oral dosage form comprising 24 mg of deutetrabenazine provides an in vivo plasma profile for total α- and β-dihydrodeutetrabenazine that includes a geometric mean AUC0-24 of about 400,000 to 800,000 h*pg/mL at steady state and/or a mean Cmax of less than about 40,000 pg/mL at steady state.

In one embodiment, administration of the oral dosage form comprising 36 mg of deutetrabenazine provides an in vivo plasma profile for total α- and β-dihydrodeutetrabenazine that includes a geometric mean AUC0-24 of about 615,000 to 1,200,000 h*pg/mL at steady state and/or a mean Cmax of less than about 60,000 pg/mL at steady state.

In one embodiment, administration of the oral dosage form comprising 48 mg of deutetrabenazine provides an in vivo plasma profile for total α- and β-dihydrodeutetrabenazine that includes a geometric mean AUC0-24 of about 800,000 to 1,600,000 h*pg/mL at steady state and/or a mean Cmax of less than about 80,000 pg/mL at steady state.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 6 mg of deutetrabenazine microparticles, provides an in vivo plasma profile for total α- and β-dihydrodeutetrabenazine that includes a geometric mean AUC_{0-inf} of about 90,000 to 142,750 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 6 mg of deutetrabenazine microparticles, provides an in vivo plasma profile for total α- and β-dihydrodeutetrabenazine that includes a geometric mean Cmax of less than about 4,600 pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 12 mg of deutetrabenazine microparticles, provides an in vivo plasma profile for total α- and β-dihydrodeutetrabenazine that includes a geometric mean AUC_{0-inf} of about 180,00 to 285,500 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 12 mg of deutetrabenazine microparticles, provides an in vivo plasma profile for total α- and β-dihydrodeutetrabenazine that includes a geometric mean Cmax of less than about 9,200 pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 24 mg of deutetrabenazine microparticles, provides an in vivo plasma profile for total α- and β-dihydrodeutetrabenazine that includes a geometric mean AUC_{0-inf} of about 360,000 to 571,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 24 mg of deutetrabenazine microparticles, provides an in vivo plasma profile for total α- and β-dihydrodeutetrabenazine that includes a geometric mean Cmax of less than about 18,400 pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 36 mg of deutetrabenazine microparticles, provides an in vivo plasma profile for total α- and β-dihydrodeutetrabenazine that includes a geometric mean AUC_{0-inf} of about 540,000 to 856,500 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 36 mg of deutetrabenazine microparticles, provides an in vivo plasma profile for total α- and β-dihydrodeutetrabenazine that includes a geometric mean Cmax of less than about 27,600 pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 48 mg of deutetrabenazine microparticles, provides an in vivo plasma profile for total α- and β-dihydrodeutetrabenazine that includes a geometric mean AUC_{0-inf} of about 720,000 to 1,142,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein single dose administration of the multiparticulate dosage form, which comprises a total amount of 48 mg of deutetrabenazine microparticles, provides an in vivo plasma profile for total α- and β-dihydrodeutetrabenazine that includes a geometric mean Cmax of less than about 36,800 pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 6 mg of deutetrabenazine microparticles, provides an in vivo plasma profile for total α- and β-dihydrodeutetrabenazine at steady state that includes a mean AUC₀₋₂₄ of about 102,500 to 200,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 6 mg of deutetrabenazine microparticles provides an in vivo plasma profile for total α- and β-dihydrodeutetrabenazine at steady state that includes a mean Cₘₐₓ of less than about 10,000 pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 12 mg of deutetrabenazine microparticles provides an in vivo plasma profile for total α- and β-dihydrodeutetrabenazine at steady state that includes a mean AUC₀₋₂₄ of about 205,000 to 400,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 12 mg of deutetrabenazine microparticles, provides an in vivo plasma profile for total α- and β-dihydrodeutetrabenazine at steady state that includes a mean Cₘₐₓ of less than about 20,000 pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 24 mg of deutetrabenazine microparticles, provides an in vivo plasma profile for total α- and β-dihydrodeutetrabenazine at steady state that includes a mean AUC₀₋₂₄ of about 410,000 to 800,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 24 mg of deutetrabenazine microparticles, provides an in vivo plasma profile for total α- and β-dihydrodeutetrabenazine at steady state that includes a mean Cₘₐₓ of less than about 40,000 pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 36 mg of deutetrabenazine microparticles, provides an in vivo plasma profile for total α- and β-dihydrodeutetrabenazine at steady state that includes a mean AUC₀₋₂₄ of about 615,000 to 1,200,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 36 mg of deutetrabenazine microparticles, provides an in vivo plasma profile for total α- and β-dihydrodeutetrabenazine at steady state that includes a mean Cₘₐₓ of less than about 60,000 pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 48 mg of deutetrabenazine microparticles, provides an in vivo plasma profile for total α- and β-dihydrodeutetrabenazine at steady state that includes a mean AUC₀₋₂₄ of about 820,000 to 1,600,000 h*pg/mL.

In one embodiment, the invention provides a method of treating a hyperkinetic movement disorder in a subject in need thereof comprising orally administering to the subject a once daily multiparticulate dosage form according to any one of the embodiments of the invention wherein the multiparticulate dosage form which comprises a total amount of 48 mg of deutetrabenazine microparticles, provides an in vivo plasma profile for total α- and β-dihydrodeutetrabenazine at steady state that includes a mean Cₘₐₓ of less than about 80,000 pg/mL.

In one embodiment, the invention provides a method of treating a hyperkinetic movement disorder comprising: administering a multiparticulate dosage form according to any one of the embodiments of the invention, wherein not more than 15% of the drug formulation is released after 2 hours when tested in 500 mL acid phosphate buffer at pH 3.0 using a USP II dissolution apparatus.

In one embodiment, the invention provides a method of treating a hyperkinetic movement disorder in a subject in need thereof comprising: administering to the subject a once daily multiparticulate dosage form according to any one of the embodiments of the invention, wherein not more than 60 wt % of the drug formulation is released within 8 hours when tested in 500 mL acid phosphate buffer at pH 3.0 using a USP II dissolution apparatus.

In some embodiments, the invention provides a method of treating a hyperkinetic movement disorder comprising: administering a multiparticulate dosage form according to any one of the embodiments of the invention, wherein not more than 15 wt % of the drug formulation is released after 2 hours and wherein 40-60 wt % of the drug formulation is released within 7 or 8 hours when tested in 500 mL acid phosphate buffer at pH 3.0 using a USP II dissolution apparatus.

Further provided herein is a process for manufacturing the immediate release beads or the core of the sustained release beads, comprising the steps of
a) providing a dispersion of nanonized deutetrabenazine with a pharmaceutically acceptable excipient, wherein the pharmaceutically acceptable excipient comprises: an antioxidant, a binder, an anti-foaming agent, a filler, and a surfactant;
b) forming immediate release granules, immediate release pellet or immediate release tablet from the dispersion of a); or coating an inert particle with the dispersion of a); thereby generating the immediate release beads or the core of the sustained release beads, respectively.

Further provided is a process for manufacturing the sustained release beads comprising the steps of
a) providing a core, wherein the core comprises immediate release granules, immediate release pellet or immediate release tablet comprising a dispersion of deutetrabenazine and a pharmaceutically acceptable excipient; or an inert particle coated with a dispersion of deutetrabenazine and a pharmaceutically acceptable excipient;
c) coating the core of a) with a pH-independent polymer coating, a pH-dependent polymer coating or with a pH-independent polymer coating and a pH-dependent polymer coating; thereby generating sustained release beads.

In some embodiments of the core or immediate release particles, the pharmaceutically acceptable excipient comprises: an antioxidant comprising butylated hydroxyanisole and butylated hydroxytoluene NF, a water-soluble binder comprising hydroxypropyl cellulose, an anti-foaming agent comprising simethicone, a filler comprising lactose monohydrate and sodium bicarbonate, and a surfactant comprising sodium lauryl sulfate.

The dosage form may be manufactured by loading a capsule shell or a sachet with a population of sustained release beads comprising a core and a pH-independent coating.

The dosage form may be manufactured by loading a capsule shell or a sachet with a population of immediate release beads and a population of sustained release beads comprising a core and a pH-dependent coating, the pH-dependent coating targeting the small intestine.

The dosage form may be manufactured by loading a capsule shell or a sachet with a population of immediate release beads and a population of sustained release beads comprising a core and a pH-dependent coating, the pH-dependent coating targeting the large intestine/colon.

The dosage form may be manufactured by loading a capsule shell or a sachet with a population of immediate release beads, a population of sustained release beads comprising a core and a pH-dependent coating targeting the small intestine and a population of sustained release beads comprising a core and a pH-dependent coating targeting the large intestine/colon.

Further provided is nanonized deutetrabenazine having a median particle size or about 0.02 to about 2.0 micron. In some embodiments, the nanonized deutetrabenazine has a particle size distribution characterized by a D90 of about 0.8 to 1.6 micron, a D50 of about 0.2 to about 0.6 micron and a D10 of about 0.1 to about 0.2 micron.

### EXAMPLES

The following examples are provided to supplement the prior disclosure and to provide a better understanding of the subject matter described herein. These examples should not be considered to limit the described subject matter. It is understood that the examples and embodiments described herein are for illustrative purposes only and that various modifications or changes in light thereof will be apparent to persons skilled in the art and are to be included within, and can be made without departing from, the true scope of the disclosure.

### Example 1—Manufacturing Process Development

The manufacturing process for the multiparticulate dosage form includes the following steps:
a. Manufacturing of deutetrabenazine dispersion
b. Coating of particles with deutetrabenazine dispersion to generate deutetrabenazine coated particles or manufacture of core granules/pellets/tablets from deutetrabenazine dispersion;
c. Sustained release coating of the deutetrabenazine particles;
d. Optional packaging/encapsulation

1. Drug Substance Physical Characterization

The deutetrabenazine particle size distributions following manufacture (untreated), micronization (air jet mill) and nano-milling (Dyno bead mill) are shown in Table 1.

TABLE 1
Particle Size Distribution (PSD) of
unmilled and milled drug substance

| PSD | unmilled | micro-milled | nano-milled |
|---|---|---|---|
| D10 (μm) | 9.08 | 1.08 | 0.07 |
| D50 (μm) | 59.66 | 3.31 | 0.14 |
| D90 (μm) | 213.07 | 7.05 | 0.34 |

Using a Mastersizer 3000 (Malvern Instruments), the following settings were used for dry measurement of the micro-milled and unmilled deutetrabenazine:

| Analysis model | Mie |
|---|---|
| Obscuration | 1.12% |
| Sample measurement time | 24 sec |

Using a Mastersizer 3000 (Malvern Instruments), the following settings were used for wet measurement of the nano-milled deutetrabenazine:

| Analysis model | Mie |
|---|---|
| Obscuration | 7% |
| Sample measurement time | 6 sec |
| Dispersant | Water |

Deutetrabenazine is a weakly basic compound with relatively good solubility in acidic environment (pH<4) and poorly soluble at pH ≥4 (<2.3 mg/mL). Deutetrabenazine is permeable through the small intestinal (SI) segments in a rat perfusion model. In humans, approximately 80% of a radioactive dose was recovered in urine in a human [¹⁴C]-AME study with deutetrabenazine dosed as powder in capsule (PIC) suggesting that the compound is well-absorbed in the small intestine and large intestine/colon. Levels of absorption of deutetrabenazine in the lower GI in rats was shown to be Jejunum<Mid-small intestine<Colon<Ileum, with ileum absorption about 0.0006 cm/sec.

The dosage forms disclosed herein were developed to achieve similar pharmacokinetics (PK) of 2 doses of AUSTEDO® 12 mg tablets with a single daily dose (QD). Drug solubility at pH greater >3 was tested in un-milled drug substance (DS) batches, compared to micro-milled and nano-milled DS batches.

For all three batches, the deutetrabenazine was dispersed in an excipient solution of butylated hydroxytoluene (antioxidant), butylated hydroxyanisole (antioxidant), hypromellose 2910 (hydroxypropyl methylcellulose, binder), lactose monohydrate (filler), sodium lauryl sulfate (surfactant), sodium bicarbonate (filler) and water. Simethicon 30% emulsion was added to avoid foaming during the process. For samples 1, 2 and 3, un-milled deutetrabenazine, deutetrabenazine with a particle size of D90<10 micron, and deutetrabenazine with a particle size of D90<1 micron were used, respectively. All three dispersions were sprayed on 25/30 sugar spheres using a GPCG-2 Wurster processing unit. Table 2 shows compositions of the deutetrabenazine-coated spheres of Samples 1, 2 and 3.

Deutetrabenazine coated particles were encapsulated in Size 0 capsule shells and dissolution was performed in 500 mL Phosphate buffer pH 6.8, USP-II apparatus, 75 rpm. Samples were collected at 10, 20, 30, 40, 60, 80, 100 and 120 minutes.

TABLE 2
Composition of Samples 1, 2 and 3.

| | Sample 1 | Sample 2 | Sample 3 |
|---|---|---|---|
| deutetrabenazine (un-milled) | 10.52% | | |
| deutetrabenazine (micro-milled) | | 10.52% | |
| deutetrabenazine (nano-milled) | | | 10.52% |
| Butylated Hydroxytoluene | 0.175% | 0.175% | 0.175% |
| Butylated Hydroxyanisole | 0.438% | 0.438% | 0.438% |
| Hydroxypropyl Cellulose, NF | 2.630% | 2.630% | 2.630% |
| (Klucel LF) | | | |
| Simethicone 30% Emulsion, USP | 0.877% | 0.877% | 0.877% |
| Lactose Monohydrate, NF | 12.714% | 12.714% | 12.714% |
| (Impalpable) | | | |
| Sodium Lauryl Sulfate, NF | 2.060% | 2.060% | 2.060% |
| Sodium Bicarbonate, USP (Grade 1) | 0.438% | 0.438% | 0.438% |
| Lactose Monohydrate, NF | 5.261% | 5.261% | 5.261% |
| (Impalpable) | | | |
| Sodium Lauryl Sulfate, NF | 3.507% | 3.507% | 3.507% |
| Purified Water, USP | q.s. | q.s. | q.s. |
| Sugar Spheres 25/30 | 61.377% | 61.377% | 61.377% |

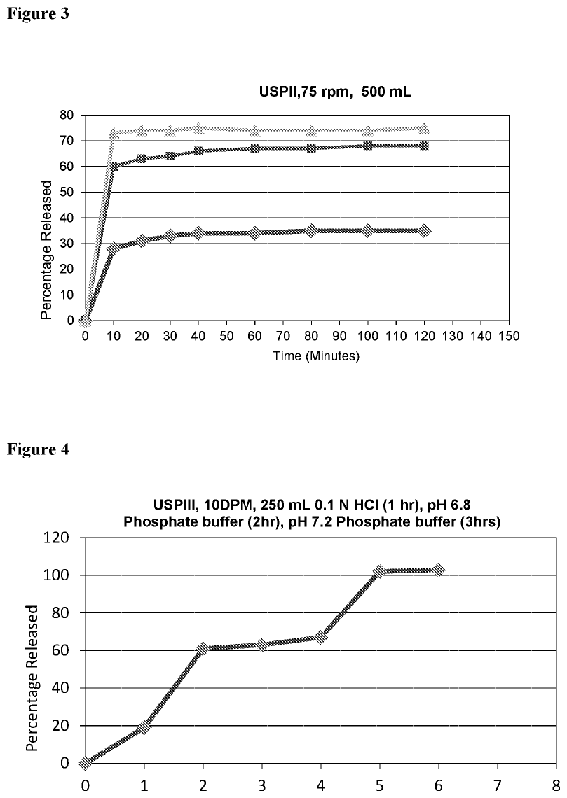

FIG. 3 shows the dissolution profiles of Samples 1, 2, and 3 in 500 mL Phosphate Buffer pH 6.8, USPII apparatus, 75 rpm. The micro-milled and nano-milled particles exhibit a better dissolution profile in pH 6.8 than un-milled sample. The diamonds represent release profile of the unmilled sample, showing poor release (˜30-35 wt % even after 2 hr); the squares represent the release profile of the micro-milled deutetrabenazine and the triangles represent the release profile of the nano-milled deutetrabenazine.

### Example 2: Milling of Deutetrabenazine

Deutetrabenazine particle size was reduced to a nanometer size (<1.0 micron) using a wet Dyno milling process in several passes. The milled deutetrabenazine was dispersed in in excipient as above. The dispersion was passed through a 0.3 liter agitator bead mill containing 950 g of very high density zirconium oxide beads. The dispersion was passed through the agitator mill for up to 2 hrs to obtain particle size below 1.0 micron. Table 3 shows PSD of the dispersion after different milling times.

TABLE 3
Particle size distribution following different milling times

| Passes through Mill | | D₁₀ | | D₅₀ | | D₉₀ | |
|---|---|---|---|---|---|---|---|
| Unmilled | | 78.407 | μm | 268.32 | μm | 554.679 | μm |
| 1 | pass | 0.801 | μm | 2.485 | μm | 9.906 | μm |
| 5 | pass | 0.263 | μm | 1.234 | μm | 3.208 | μm |
| 30 | minutes | 0.190 | μm | 0.558 | μm | 1.585 | μm |
| 60 | minutes | 0.174 | μm | 0.348 | μm | 1.132 | μm |
| 90 | minutes | 0.166 | μm | 0.288 | μm | 0.893 | μm |
| 120 | minutes | 0.166 | μm | 0.284 | μm | 0.863 | μm |

Following milling, a solution of lactose and sodium lauryl sulfate was prepared and added to the nano milled dispersion and mixed for 30 minutes using an air mixer. The resultant deutetrabenazine suspension was sprayed on sugar spheres using a Glatt fluid bed coater to generate deutetrabenazine-coated particles. A first portion of the deutetrabenazine coated particles was left as is (i.e. immediate release population); a second portion was further coated with a sustained release coating (methacrylic acid and ethyl acrylate copolymer dispersion, pH5.5-7); and a third portion was coated with a second sustained release coating (methacrylic acid and methyl methacrylate copolymer dispersion pH>7). The total composition of the three particle populations in the dosage form is provided below, in Table 4.

TABLE 4

| Deutetrabenazine Coated Particles | |
|---|---|
| (Core or Immediate Release beads) | |
| deutetrabenazine | 11.99% |
| Sugar spheres 25/30 | 55.97% |
| Butylated hydroxytoluene | 0.2% |
| Butylated hydroxyanisole | 0.5% |
| Hydroxypropyl Cellulose, NF | 3.0% |
| (Klucel LF) | |
| Simethicone 30% Emulsion, USP | 1.0% |
| Lactose Monohydrate, NF | 14.49% |
| (Impalpable) | |
| Sodium Lauryl Sulfate, NF | 2.349% |
| Sodium Bicarbonate, USP | 0.5% |
| (Grade 1) | |
| Lactose Monohydrate, NF | 6.0% |
| (Impalpable) | |
| Sodium Lauryl Sulfate, NF | 4.0% |
| Purified Water, USP | q.s. |

| Sustained Release | | Sustained Release | |
|---|---|---|---|
| Particles (pH 5.5-7) | | Particles (pH > 7) | |
| coated particles (above) | 71.17% | coated particles (above) | 65.70% |
| Methacrylic Acid-Ethyl | 21.35% | Methacrylic Acid-Methyl | 19.71% |
| Acrylate Copolymer NF | | Methacrylate Copolymer, | |
| | | NF | |
| Triethyl Citrate, NF | 2.14% | Triethyl Citrate, NF | 5.72% |
| Talc USP | 5.34% | Talc USP | 8.87% |
| Purified Water, USP | q.s. | Isopropyl Alcohol, USP | q.s. |
| | | Acetone, NF | q.s. |
| | | Purified Water, USP | q.s. |

The immediate release particles and the two populations of the sustained release particles were filled into a capsule shell. Dissolution of the filled capsule was performed in a USPIII apparatus at 10 dpm. The pH values in the apparatus were selected based on the pH of the GI. A pH gradient was 0-1 hr in 0.1N HCl, 1 hr-3 hrs in phosphate buffer pH 6.8 and 3 hrs-6 hrs in phosphate buffer pH 7.2. Samples were collected at 1, 2, 3, 4, 5 and 6 hrs time points. FIG. 4 shows the resulting dissolution profile across the pH gradients.

### Example 3: Immediate Release and Sustained Release Dosage Form

The composition of dosage forms comprising sustained release beads comprising deutetrabenazine coated inert particles coated with a pH-independent polymer coating is provided in Table 5 as Samples 5-10.

TABLE 5

| | mg/Capsule | | |
|---|---|---|---|
| Ingredients | Samples 5, 6 | Samples 7, 8 | Samples 9, 10 |
| deutetrabenazine (Nano milled) | 24.00 | 24.00 | 24.00 |
| Butylated Hydroxyanisole, NF | 1.00 | 1.00 | 1.00 |
| Butylated Hydroxytoluene, NF | 0.40 | 0.40 | 0.40 |
| Hydroxypropyl Cellulose, NF (Klucel LF) | 6.00 | 6.00 | 6.00 |
| Simethicone 30% Emulsion, USP | 2.0 | 2.0 | 2.0 |
| Lactose Monohydrate, | 29.00 | 29.00 | 29.00 |
| NF (Impalpable)-Part A | | | |
| Lactose Monohydrate, | 12.00 | 12.00 | 12.00 |
| NF (Impalpable)-Part B | | | |
| Sodium Lauryl Sulfate, NF-Part A | 4.70 | 4.70 | 4.70 |
| Sodium Lauryl Sulfate, NF-Part B | 8.00 | 8.00 | 8.00 |
| Sodium Bicarbonate, USP (Grade 1) | 1.00 | 1.00 | 1.00 |
| Purified Water, USP-Part A^{#} | q.s. | q.s. | q.s. |
| Purified Water, USP-Part B^{#} | q.s. | q.s. | q.s. |
| Sugar Spheres, NF 25/30 | 112.00 | 112.00 | 112.00 |
| (600/710, Suglets # PF008) | | | |
| Total | 200.2 | 200.2 | 200.2 |

pH-independent polymer coating

| | | | | | | |
|---|---|---|---|---|---|---|
| Coating level | 20% | 40% | 20% | 40% | 20% | 40% |
| Cellulose Acetate, NF 398-10 | 36.0 | 72.0 | 38.0 | 76.0 | 20.0 | 40.0 |
| Cellulose Acetate 320S | n/a | n/a | n/a | n/a | 12.0 | 24.0 |
| Polyethylene Glycol 3350 | 4.0 | 8.0 | 2.00 | 4.0 | 8.0 | 16.0 |
| Purified Water | q.s | q.s | q.s | q.s | q.s | q.s |
| Acetone | q.s | q.s | q.s | q.s | q.s | q.s |

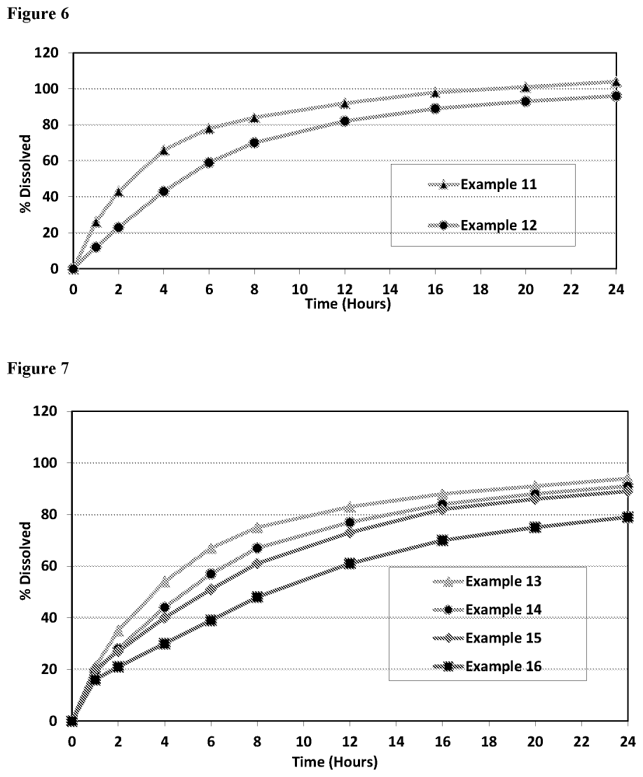

Nano milled active coated pellets were coated with a cellulose acetate and polyethylene glycol coating. The extended release particles were filled in capsules and drug release profile was evaluated in pH 3.0 using USP II at 75 RPM up to 24 hours, graph shown in FIG. 6.

Samples 11-12 are provided in Table 6.

TABLE 6

| | mg/Capsule |
|---|---|
| Ingredients | Samples 11 and 12 |
| Deu-TBZ (Nano milled) | 24.00 |
| Butylated Hydroxyanisole, NF | 1.00 |
| Butylated Hydroxytoluene, NF | 0.40 |
| Hydroxypropyl Cellulose, NF (Klucel LF) | 6.00 |
| Simethicone 30% Emulsion, USP | 2.0 |
| Lactose Monohydrate, NF (Impalpable)-Part A | 29.00 |
| Lactose Monohydrate, NF (Impalpable)-Part B | 12.00 |
| Sodium Lauryl Sulfate, NF- Part A | 4.70 |
| Sodium Lauryl Sulfate, NF- Part B | 8.00 |
| Sodium Bicarbonate, USP (Grade 1) | 1.00 |
| Purified Water, USP-Part A^{#} | q.s. |
| Purified Water, USP-Part B^{#} | q.s. |
| Sugar Spheres, NF 60/80 (Contains Cornstarch) | 56.0 |
| Total | 144.1 |

pH-independent polymer coating

| Ethylcellulose:Polyethylene Glycol 3350 | 70:30 | |
|---|---|---|
| Coating level | 20% | 30% |
| Ethylcellulose, NF 10 cps | 20.16 | 30.24 |
| Polyethylene Glycol 3350 | 8.64 | 12.96 |
| Triethyl Citrate | 3.02 | 4.54 |
| Talc | 14.40 | 21.60 |
| Ethyl Alcohol 190 proof | q.s | q.s |
| Purified Water | q.s | q.s |

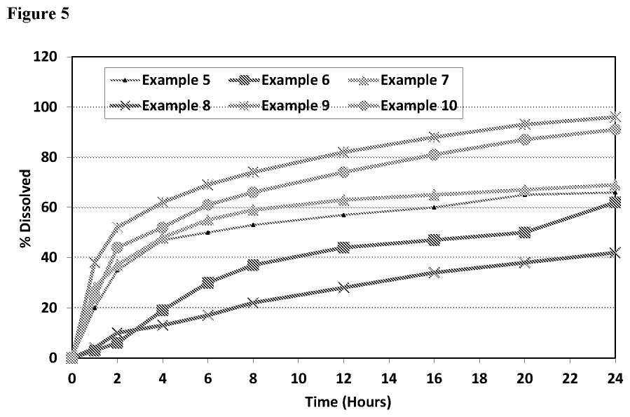

API coated particles were further coated with ethylcellulose and polyethylene glycol. The sustained release beads were filled in capsule shells and drug release profile was evaluated in pH 3.0 using USP II at 75 RPM up to 24 hours, graph shown in FIG. 5.

Samples 13-16 are provided in Table 7.

TABLE 7

| | mg/Capsule | |
|---|---|---|
| Ingredients | Samples 13, 14 | Sample 15, 16 |
| DEU-TBZ (Nano milled) | 24.00 | 24.00 |
| Butylated Hydroxyanisole, NF | 1.00 | 1.00 |
| Butylated Hydroxytoluene, NF | 0.40 | 0.40 |
| Hydroxypropyl Cellulose, NF | 6.00 | 6.00 |
| (Klucel LF) | | |
| Simethicone 30% Emulsion, USP | 2.0 | 2.0 |
| Lactose Monohydrate, NF | 29.00 | 29.00 |
| (Impalpable)-Part A | | |
| Lactose Monohydrate, NF | 12.00 | 12.00 |
| (Impalpable)-Part B | | |
| Sodium Lauryl Sulfate, NF- Part A | 4.70 | 4.70 |
| Sodium Lauryl Sulfate, NF- Part B | 8.00 | 8.00 |
| Purified Water, USP-Part A^{#} | q.s. | q.s. |
| Purified Water, USP-Part B^{#} | q.s. | q.s. |
| Sugar Spheres, NF 25/30 | 56.0 | 56.0 |
| (600/710, Suglets # PF008) | | |
| Total | 143.1 | 143.1 |

pH-independent polymer coating

| | | | | |
|---|---|---|---|---|
| Coating level | 20% | 30% | 20% | 30% |
| Ethylcellulose, NF 10 cps | 22.88 | 34.32 | 22.88 | 34.32 |
| Polyethylene Glycol 3350 | n/a | n/a | 5.72 | 8.58 |
| Povidone K 30 | 5.72 | 8.58 | n/a | n/a |
| Triethyl Citrate | 3.43 | 5.15 | 3.43 | 5.15 |
| Talc | 14.30 | 21.45 | 14.30 | 21.45 |
| Ethyl Alcohol 190 proof | q.s. | q.s. | q.s. | q.s. |
| Purified Water | q.s. | q.s. | q.s. | q.s. |

Deutetrabenazine-coated particles were further coated with ethylcellulose and polyethylene glycol coating. The sustained release beads were filled in capsules and drug release profile was evaluated in pH 3.0 using USP II at 75 RPM up to 24 hours, graph shown in FIG. 7. These data show a release profile of about 40-60 wt % at 7 hours.

### Example 4—Single Dose Bioavailability Assessment

Microparticulate dosage forms containing deutetrabenazine are produced as disclosed in Example 1 and studied in a single dose pharmacokinetic study.

The primary objective is to assess the comparative bioavailability (BA) of deutetrabenazine and deuterated α- and β-dihydrotetrabenazine (deuHTBZ) metabolites following a single administration of the microparticulate dosage form (Test) compared to a single 12 mg Austedo® tablet administered twice, 12 hours apart (bid), under fasted conditions.

Study Population and Number of Subjects: The study includes healthy male and female non-smoking subjects.

Duration of Subject Participation: The study includes a screening period of 2-4 weeks (period 1), an open label treatment period with the test dosage forms (Test) and the reference formulation (Ref) (period 2), and a follow-up visit at least 1 day later (period 3).

Treatments:

Treatment sequence A:

Day 1—administration of Test.

Days 2-3—at least 6 hours wash out of Test followed by administration of Ref.

Treatment sequence B:

Day 1—administration of Ref

Days 2-3—at least 6 hours wash out of Ref, followed by administration of Test.

The primary objective was addressed using the following parameters:
maximum observed concentration (Cmax)
area under the plasma concentration-time (AUC) from time 0 to the time of the last measurable plasma concentration (AUC0-t)
AUC extrapolated to infinity (AUC0-∞)
AUC from time 0 to 24 hours post dose (AUC0-24 h) Analyses

AUC0-t, AUC0-∞, and AUC0-24 h are calculated using the trapezoidal rule. The Cmax, AUC0-t, AUC0-∞, and AUC0-24 h data are natural log-transformed prior to the statistical analysis. Comparisons of Cmax, AUC0-t, AUC0-∞, and AUC0-24 h between treatments (T2A vs R) will be carried out using a separate parametric analysis of variance (ANOVA) model with fixed effect terms for sequence, period, treatment group, and a random effect of subject within sequence. The difference between the reference formulation (Ref) and the test formulation (Test) will be evaluated by constructing 90% confidence intervals for the Test/Ref ratios, based on the least-square means from the ANOVA for the log-transformed Cmax, AUC0-t, AUC0-∞ and AUC0-24 h. The treatment difference and the associated 90% confidence interval estimated from the ANOVA on the log scale will be back-transformed to obtain the estimated ratio of geometric means between treatment groups and the 90% confidence interval for this ratio.

### Results

The once-daily dose of Test dosage forms provide similar deuHTBZ plasma concentrations observed for the Ref. The multiparticulate dosage forms disclosed herein are administered once daily and provide a similar treatment effect to that of AUSTEDO and also have no safety concerns.

### Example 5—Multiple Dose Bioavailability Assessment

The multiparticulate dosage forms containing 24 mg of deutetrabenazine were produced as disclosed in Example 1 and are studied in an open label, randomized, multiple-dose, 2-way crossover study in healthy volunteers.

The primary objective is to assess the bioequivalence (BE) of administration of Test, once daily (qd) compared to bid administration of Ref, under fasted or fed conditions.

Treatment includes 7 days repeated dosing of Test once daily versus 7 days repeated dosing of Ref, bid.

AUCt, Cₘₐₓ, tₘₐₓ, Cₘᵢₙ, Cₐᵥ for deutetrabenazine and deuHTBZ are analyzed, at steady state.

### Results

Multiple dosing of Test has comparable pK parameters to that of Ref, at steady state. Therefore similar efficacy response is expected with once daily administration, having no safety concerns.

### Example 6: Food Effect Study

Multiparticulate dosage forms containing 24 mg deutetrabenazine are produced as disclosed in Example 1 and studied in an open label, randomized, two-way crossover study, to assess the comparative bioavailability of deutetrabenazine and deuHTBZ in the fed compared to the fasted state, following a single administration of 24 mg, once daily (qd) multiparticulate formulation.

Treatment includes:
A—24 mg, once daily (qd) multiparticulate formulation given as a single oral dose with water after an overnight fast of at least 10 hours.
B—24 mg, once daily (qd) multiparticulate formulation given as a single oral dose with water, 30 minutes after the start of standardized high calorie, high fat breakfast administered after an overnight fast of at least 10 hours.

Subject will receive treatments A/B with at least 6 days washout period.

AUCt, Cmax, tmax, Cmin, Cav for deutetrabenazine and deuHTBZ will be analyzed.

### Results

The similar plasma concentrations of deutetrabenazine and deuHTBZ, following single administration with or without food, show that the multiparticulate dosage from can be administered regardless of food.

All patents, patent applications, and publications mentioned in the specification are indicative of the levels of those of ordinary skill in the art to which the invention pertains. All patents, patent applications, and publications are herein incorporated by reference to the same extent as if each individual publication was specifically and individually indicated to be incorporated by reference. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

For the foregoing embodiments, each embodiment disclosed herein is contemplated as being applicable to each of the other disclosed embodiments. For instance, the elements recited in the method embodiments can be used in the pharmaceutical composition, package, and use embodiments described herein and vice versa.

1. A controlled release oral dosage form for once daily administration of deutetrabenazine comprising a population of sustained release beads; wherein the sustained release beads comprise a core comprising an amount of deutetrabenazine and a pharmaceutically acceptable excipient, and further comprising a pH-independent polymer coat, a pH-dependent polymer coat or a pH-independent polymer coat further coated with a pH-dependent polymer coat.

2. The dosage form of claim 1, wherein the core comprises a) immediate release granules, immediate release pellet or immediate release tablet comprising the deutetrabenazine and the pharmaceutically acceptable excipient or b) an inert particle coated with a dispersion of the deutetrabenazine and the pharmaceutically acceptable excipient.

3. The dosage form of claim 1, further comprising a population of immediate release beads; wherein the population of immediate release beads comprises a) immediate release granules, immediate release pellet or immediate release tablet comprising an amount of deutetrabenazine and a pharmaceutically acceptable excipient or b) an inert particle coated with an amount of deutetrabenazine and a pharmaceutically acceptable excipient.

4. The dosage form of claim 3, wherein the amount of deutetrabenazine and/or the pharmaceutically acceptable excipient are identical in the core of the sustained release beads and in the immediate release beads, or wherein the amount of deutetrabenazine and/or the pharmaceutically acceptable excipient are different in the core of the sustained release beads and in the immediate release beads.

5. The dosage form of claim 1, wherein the deutetrabenazine has a median particle size of 0.02 to 2.0 micron, or 0.02 to 0.9 micron, or 0.05 to 0.5 micron, or 0.1 to 2.0 micron, or 0.1 to 1.6 micron, or 0.2 to 1.6 micron, or 0.15 to 1.2 micron, or 0.15 to 1.0 micron.

6. The dosage form of claim 5, wherein the deutetrabenazine has a particle size distribution characterized by a D90 of about 0.8 to about 1.6 micron.

7.-10. (canceled)

11. The dosage form of claim 1, wherein the pharmaceutically acceptable excipient comprises any one of an antioxidant, a binder, a filler, a surfactant, an anti-foaming agent or combinations thereof.

12.-30. (canceled)

31. The dosage form of claim 1, wherein the sustained release bead comprises a pH-independent polymer coat coating the core.

32.-35. (canceled)

36. The dosage form of claim 1, wherein the sustained release beads comprise a pH-dependent polymer coat coating the core.

37.-48. (canceled)

49. The dosage form of claim 1, wherein the dosage form comprises a total of 6 mg-72 mg of deutetrabenazine.

50.-55. (canceled)

56. The dosage form of claim 1, comprising a population of sustained release beads and further comprising a population of immediate release beads, wherein the immediate release beads comprise a) immediate release granules, immediate release pellet or immediate release tablet comprising an amount of deutetrabenazine and a pharmaceutically acceptable excipient or b) an inert particle coated with an amount of deutetrabenazine and a pharmaceutically acceptable excipient.

57.-66. (canceled)

67. The dosage form of claim 1, wherein about 40 wt %-60 wt % of deutetrabenazine is released within 7 hours, as measured in a USPII dissolution device, pH pH3.0 phthalate buffer, 75 rpm.

68. (canceled)

69. A method of treating a VMAT2 mediated disorder comprising, orally administering to a patient in a need thereof, the controlled release dosage form of claim 1.

70.-72. (canceled)

73. The dosage form or the method of claim 1, wherein single dose administration of the oral dosage form comprising 6 mg of deutetrabenazine provides an in vivo plasma profile for total α- and β-dihydrodeutetrabenazine that includes a geometric mean AUC0-inf of about 90,000 to 142,750 h*pg/mL and/or a geometric mean Cmax of less than about 4,600 pg/mL; or
wherein single dose administration of the oral dosage form comprising 12 mg of deutetrabenazine provides an in vivo plasma profile for total α- and β-dihydrodeutetrabenazine that includes a geometric mean AUC0-inf of about 180,000 to 285,500 h*pg/mL and/or a geometric mean Cmax of less than about 9,200 pg/mL; or
wherein single dose administration of the oral dosage form comprising 24 mg of deutetrabenazine provides an in vivo plasma profile for total α- and β-dihydrodeutetrabenazine that includes a geometric mean AUC0-inf of about 360,000 to 571,000 h*pg/mL and/or a geometric mean Cmax of less than about 18,400 pg/mL; or
wherein single dose administration of the oral dosage form comprising 36 mg of deutetrabenazine provides an in vivo plasma profile for total α- and β-dihydrodeutetrabenazine that includes a geometric mean AUC0-inf of about 540,000 to 856,500 h*pg/mL and/or a geometric mean Cmax of less than about 27,600 pg/mL; or
wherein single dose administration of the oral dosage form comprising 48 mg of deutetrabenazine provides an in vivo plasma profile for total α- and β-dihydrodeutetrabenazine that includes a geometric mean AUC0-inf of about 720,000 to 1,142,000 h*pg/mL and/or a geometric mean Cmax of less than about 36,800 pg/mL.

74.-77. (canceled)

78. The dosage form or the method of claim 1, wherein administration of the oral dosage form comprising 6 mg of deutetrabenazine provides an in vivo plasma profile for total α- and β-dihydrodeutetrabenazine that includes a geometric mean AUC0-24 of about 102,500 to 200,000 h*pg/mL at steady state and/or a mean Cmax of less than about 10,000 pg/mL at steady state; or,
wherein administration of the oral dosage form comprising 12 mg of deutetrabenazine provides an in vivo plasma profile for total α- and β-dihydrodeutetrabenazine that includes a geometric mean AUC0-24 of about 205,000 to 400,000 h*pg/mL at steady state and/or a mean Cmax of less than about 20,000 pg/mL at steady state; or,
wherein administration of the oral dosage form comprising 24 mg of deutetrabenazine provides an in vivo plasma profile for total α- and β-dihydrodeutetrabenazine that includes a geometric mean AUC0-24 of about 400,000 to 800,000 h*pg/mL at steady state and/or a mean Cmax of less than about 40,000 pg/mL at steady state; or,
wherein administration of the oral dosage form comprising 36 mg of deutetrabenazine provides an in vivo plasma profile for total α- and β-dihydrodeutetrabenazine that includes a geometric mean AUC0-24 of about 615,000 to 1,200,000 h*pg/mL at steady state and/or a mean Cmax of less than about 60,000 pg/mL at steady state; or,
wherein administration of the oral dosage form comprising 48 mg of deutetrabenazine provides an in vivo plasma profile for total α- and β-dihydrodeutetrabenazine that includes a geometric mean AUC0-24 of about 800,000 to 1,600,000 h*pg/mL at steady state and/or a mean Cmax of less than about 80,000 pg/mL at steady state.

79.-82. (canceled)

83. A process for manufacturing a core of the sustained release bead or the immediate release bead of a dosage form according to claim 3, comprising the steps of
a) providing a dispersion of nanonized deutetrabenazine with a pharmaceutically acceptable excipient, wherein the pharmaceutically acceptable excipient comprises: an antioxidant, a binder, an anti-foaming agent, a filler, and a surfactant;
b) forming immediate release granules, immediate release pellet or immediate release tablet from the dispersion of a); or coating an inert particle with the dispersion of a);
thereby generating the immediate release beads or the core of the sustained release beads, respectively.

84. A process for manufacturing the sustained release beads of a dosage form according to claim 1, comprising the steps of
a) providing a core, wherein the core comprises immediate release granules, immediate release pellet or immediate release tablet comprising a dispersion of deutetrabenazine and a pharmaceutically acceptable excipient; or an inert particle coated with a dispersion of deutetrabenazine and a pharmaceutically acceptable excipient;
b) coating the core of a) with a pH-independent polymer coating, a pH-dependent polymer coating or with a pH-independent polymer coating and a pH-dependent polymer coating;
thereby generating sustained release beads.

85. The process of claim 84, wherein the process for preparing the core comprises the steps of
a) providing a dispersion of nanonized deutetrabenazine with a pharmaceutically acceptable excipient, wherein the pharmaceutically acceptable excipient comprises: an antioxidant, a binder, an anti-foaming agent, a filler, and a surfactant;
b) forming immediate release granules, immediate release pellet or immediate release tablet from the dispersion of a); or coating an inert particle with the dispersion of a);
thereby generating the immediate release beads or the core of the sustained release beads, respectively.

86. (canceled)

87. The process of claim 83, wherein the nanonized deutetrabenazine is prepared by milling.

88. Nanonized deutetrabenazine comprising a median particle size of 0.02 to 2.0 micron, or 0.02 to 0.9 micron, or 0.05 to 0.5 micron, or 0.1 to 2.0 micron, or 0.1 to 1.6 micron, or 0.2 to 1.6 micron, or 0.15 to 1.2 micron, or 0.15 to 1.0 micron.

89. (canceled)
